(19) United States
(12) Patent Application Publication
(10) Pub. No.: US 2026/0347328 A1
(43) Pub. Date: Nov. 5, 2026

(54) IMPROVED PROCESS FOR PREPARING S-TRIAZINE DERIVATIVES

(71) Applicant: BASF SE, Ludwigshafen am Rhein (DE)

(72) Inventors: Peter LEIDINGER, Ludwigshafen am Rhein (DE); Thomas BERG, Ludwigshafen am Rhein (DE); Helmut KRONEMAYER, Ludwigshafen am Rhein (DE); Joerg WERLE, Ludwigshafen am Rhein (DE)

(21) Appl. No.: 19/106,281

(22) PCT Filed: Aug. 28, 2023
(86) PCT No.: PCT/EP2023/073498

§ 371 Date: Feb. 25, 2025

(30) Foreign Application Priority Data
Aug. 29, 2022 (EP) 22192660.3

(51) Int. Cl. C07D 251/70; A61K 8/49; A61Q 17/04

(52) U.S. Cl. CPC C07D 251/70; A61K 8/4966; A61Q 17/04; A61K 2800/10

(57) ABSTRACT

The present invention relates to a process for preparing an s-triazine derivative comprising reacting in a non-polar solvent a cyanuric halide with p-aminobenzoic acid esters, wherein the reaction product is purified by gas/steam stripping. Further, the present invention relates to the s-triazine derivative of high purity and/or optimum particle properties, the use thereof as a light protecting agent and a cosmetic preparation thereof.

The present invention relates to a process for preparing an s-triazine derivative comprising reacting in a non-polar solvent a cyanuric halide with p-aminobenzoic acid esters, wherein the reaction product is purified by gas/steam stripping. Further, the present invention relates to the s-triazine derivative of high purity and/or optimum particle properties, the use thereof as a light protecting agent and a cosmetic preparation thereof.

S-triazine derivatives, i.e. 1,3,5-triazine derivatives, are known for their broad UV absorption efficiency, absorbing in both UVB and UVA sections with high light stability. Thus, s-triazine derivatives are used as light protecting agents, in particular in cosmetic applications and sunscreen compositions. Considering the contact with human skin in these applications, s-triazine derivatives are required in high purity. Typically, s-triazine derivatives are prepared by reacting a cyanuric halide (also referred to as “cyanuric acid halide”) with p-aminobenzoic acid esters in a non-polar solvent. To reach the desired product quality, side products and solvent residues have to be removed, which is time-consuming as well as energy and cost intensive in large-scale industrial applications. The desolventization is in particular challenging as too rapid drying at excessively high temperatures produces a product that is less soluble in the oils used in cosmetics.

EP 3 674 293 A1 discloses a process for synthesizing s-triazine derivatives comprising the step of reacting a cyanuric halide with a p-aminobenzoic acid ester and subsequently purifying the product using carbon and a Hyflow™ bed. However, this procedure involves an additional adsorption step and uses large amounts of solvent, which increases complexity and costs. Especially considering large-scale applications, this procedure is economically disadvantageous.

EP 2 688 875 discloses a technically simple and economic process for synthesizing s-triazine derivatives comprising the step of reacting cyanuric halide with a p-aminobenzoic acid ester with significantly reduced amounts of solvent. However, the reached product purity shown in the laboratory example is only 90.5 area % with 0.5% by weight of secondary components.

It was therefore an object of the present invention to provide an improved process for preparing s-triazine derivatives of high purity in high yields with cost-efficient product purification.

In this regard, it was desired to provide a process, which not only ensures high purity of the product, but also high solubility and stability in cosmetic preparations. Furthermore, it was an object to provide a process, which is suitable for large-scale applications. Furthermore, the process was desired to be economically and environmentally advantageous with, e.g., low amounts of solvent and few process steps.

It has surprisingly been found that the above objectives can be achieved by the process described hereinafter and in the claims.

In one embodiment, the present invention relates to a process for preparing an s-triazine derivative of formula (I)

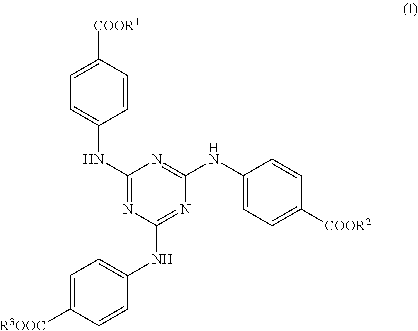

comprising reacting in a non-polar solvent
(i) a cyanuric halide; with
(ii) p-aminobenzoic acid esters

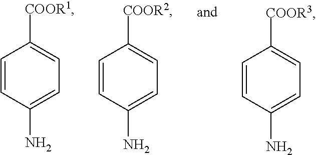

wherein
R¹, R², and R³ are each same or different and independently selected from the group consisting of hydrogen, alkali metal, ammonium, substituted ammonium, C₁-C₁₂-alkyl or polyoxyethylene; and
wherein
the reaction product is purified by gas/steam stripping.

It has surprisingly been found by the inventors of the present invention that the process of the invention provides s-triazine derivatives in high purity without requiring complex product purification. In particular, the s-triazine derivatives can be accessed in high purity and high yields by purification of the product with gas/steam stripping. In contrast to the purification methods described in the prior art, e.g. using adsorption techniques, gas/steam stripping according to the invention can easily be applied on a large industrial scale. Further, purification by gas/steam stripping prior to further purification steps like recrystallization, washing and drying decreases the impurities introduced into the solvent used for these purification steps, which is economically and ecologically advantageous in terms of subsequent purification and recycling of said solvent. In particular, the solvent used for the reaction as well as optionally side products are removed and thus do not contaminate the solvent used afterwards for the purification. Furthermore, it has been found that the product yield can be significantly increased by the process of the invention. Furthermore, the obtained s-triazine derivative has optimum physical properties in terms of, e.g., particle size, flowability as well as solubility and stability in oils used in cosmetic formulations.

This process of the invention is thus particularly advantageous regarding the combination of providing optimum product purity and yield with an effective and economic process for large scale applications.

In one embodiment of the invention, R²=R¹; and preferably R³=R²=R¹; and particularly preferably R¹, R², and R³ each represent a 2-ethylhexyl radical.

In one embodiment of the invention, the s-triazine derivative is 2,4,6-trianilino-p-(carbo-2′-ethylhexyl-1′-oxy)-1,3,5-triazine having the following chemical formula (Ia):

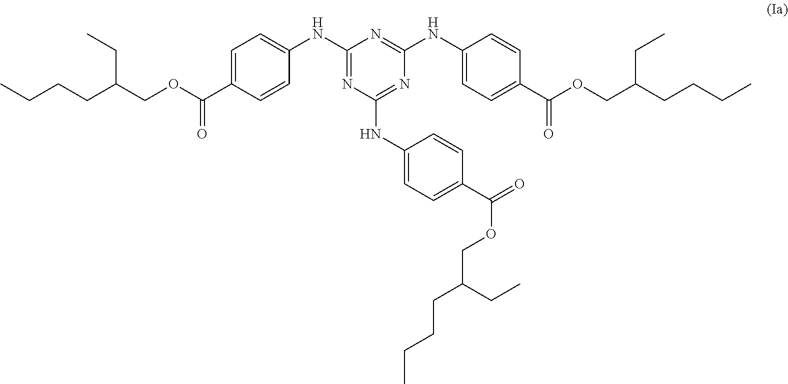 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

In one embodiment of the invention, gas/steam stripping is performed by feeding the reaction product into a vessel or reactor and stripping off the non-polar solvent and optionally side products by adding water or a polar solvent or water steam or a steam of a polar solvent or a gas or a mixture thereof to the vessel or reactor.

In one embodiment of the invention, gas/steam stripping is performed by adding water steam, ethanol steam, or nitrogen gas, preferably water steam or nitrogen gas, preferably from the bottom part of the vessel/reactor.

In one embodiment of the invention, the temperature during steam/gas stripping is less than 200 °C, preferably less than 180 °C; and/or the pressure of the steam/gas during steam/gas stripping is up to 100 bar, preferably up to 20 bar.

In one embodiment of the invention, after stripping, the s-triazine derivative is dissolved in a polar solvent, preferably a C₁-C₈-alcohol or water or a mixture thereof.

In one embodiment of the invention, after stripping, the s-triazine derivative is dissolved in a mixture of at least one C₁-C₄-alcohol with up to 10% by weight of water, preferably in a mixture of at least one C₂-C₄-alcohol with up to 10% by weight of water.

In one embodiment of the invention, the reaction is performed in an aromatic solvent, which is preferably toluene, xylene, a xylene isomeric mixture, trimethylbenzenes, a trimethylbenzene isomeric mixture, or mixtures thereof, wherein the solvent is particularly preferably azeotropically dewatered prior to the start of the reaction.

In one embodiment of the invention, the reaction is performed at a temperature of from 75 °C up to the boiling point of the used solvent for a time period of up to 48 h.

In one embodiment of the invention, the cyanuric halide is selected from cyanuric chloride, cyanuric bromide and mixtures thereof, and the cyanuric halide is preferably cyanuric chloride.

In one embodiment of the invention, the process further comprises preparing p-aminobenzoic acid-2-ethyl hexylester comprising reacting p-nitrobenzoic acid-2-ethyl hexylester with hydrogen in the presence of a catalyst.

In one embodiment of the invention, the process further comprises preparing p-nitrobenzoic acid-2-ethyl hexylester comprising reacting p-nitrobenzoic acid with 2-ethylhexanol.

In one embodiment, the present invention relates to an s-triazine derivative of formula (I), in particular 2,4,6-trianilino-p-(carbo-2′ethylhexyl-1′-oxy)-1,3,5-triazine, having a purity of at least 98% by weight, preferably having a purity of at least 99% by weight, more preferably having a purity of at least 99.5% by weight. In even more preferred embodiments, the purity may be at least 99.6, 99.7, 99.8 or 99.9% by weight. The s-triazine derivative of formula (I) may be tautomeric form I of 2,4,6-trianilino-p-(carbo-2′ethylhexyl-1′-oxy)-1,3,5-triazine (as disclosed in WO 03/074499 A1).

In one embodiment, the present invention relates to the s-triazine derivative, which is in the form of pourable or flowable particles, preferably in crystalline form, particularly preferably with an average particle diameter of from 5 μm up to 500 μm, and/or a bulk density of greater than 0.2 g/mL.

In one embodiment, the present invention relates to the s-triazine derivative, which is obtainable by the process of the present invention. In one embodiment, the s-triazine derivative of formula (I) is tautomeric form I of 2,4,6-trianilino-p-(carbo-2′ethylhexyl-1′-oxy)-1,3,5-triazine.

In one embodiment, the present invention relates to the use of the s-triazine derivative of formula (I) as a light protecting agent.

In one embodiment, the present invention relates to a cosmetic preparation comprising the s-triazine derivative of formula (I).

Preferred embodiments of the present invention can be found in the claims, the description and the examples. It is to be understood that the features mentioned above and those still to be illustrated below of the subject matter of the invention are preferred not only in the respective given combination but also in other combinations without leaving the scope of the invention.

In connection with the above embodiments of the present invention, the following definitions are provided.

In the context of the present invention the s-triazine derivative of formula (I) is obtained by reacting in a non-polar solvent a cyanuric halide with same or different p-aminobenzoic acid esters.

As used herein, the term “s-triazine derivative(s) of formula (I)” or “s-triazine derivative(s) according to the invention” or “s-triazine derivative(s)” refers to compounds of formula (I) as defined herein, i.e. 2,4,6-trisubstituted 1,3,5-triazines, wherein the substituents at the 2-, 4-, and 6-position are derived from same or different p-aminobenzoic acid esters such that these p-aminobenzoic esters are attached via their p-amino group. As indicated above, the attached substituents at the 2-, 4-, and 6-position are derived from same or different p-aminobenzoic acid esters. Thus, R¹, R² and R³ may be same or different according to the invention. It is preferred, however, that R¹=R²=R³, i.e. that the attached substituents at the 2-, 4-, and 6-position are derived from the same p-aminobenzoic acid ester.

The non-polar solvents generally include alkanes, aromatic solvents, and ethers, or mixtures thereof. Preferred non-polar solvents according to the invention are aromatic solvents and particularly preferred are toluene, xylene, a xylene isomeric mixture, trimethylbenzenes, a trimethylbenzene isomeric mixture, or a mixture thereof. Further, the solvent is preferably dewatered azeotropically prior to the start of the reaction. Residual water reduces the yield and increases the amount of side products as cyanuric halides have the tendency to hydrolyze giving rise to mono-, di-, and/or trihydroxy-triazines. In addition, under the elevated process temperatures during s-triazine derivative formation, partial or full hydrolysis of the p-aminobenzoic acid esters, intermediates, and/or the product s-triazine derivative can occur.

The term “cyanuric halide” or “cyanuric acid halide” as used herein refers to a 1,3,5-triazine substituted with the same halide in 2-, 4-, and 6-position. The cyanuric halide is preferably cyanuric bromide, cyanuric chloride, or a mixture thereof, and more preferably cyanuric chloride (CAS number: 108-77-0). Cyanuric halides are highly reactive species. The three halides at the 2-, 4-, and 6-position can be easily substituted by a nucleophilic species in order to obtain 2,4,6-trisubstituted 1,3,5-triazines. As three halides are substituted in such a reaction, cyanuric halides are typically reacted with at least three equivalents of a nucleophile. If different types of nucleophiles are used, the molar ratio of cyanuric acid to nucleophile is again typically at least 1:3, preferably from 1:3 to 1:5. However, the nucleophile may then be represented by different types of nucleophiles. The reaction may then be conducted in a step-wise manner such that the different types of nucleophiles are reacted sequentially with the cyanuric halide.

The term “p-aminobenzoic acid esters” or “4-aminobenzoic acid esters” as used herein refers to compounds of the following general formulae

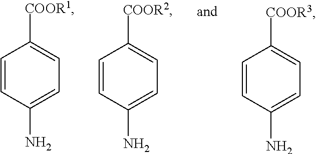

wherein R¹, R², and R³ are each same or different and independently selected from the group consisting of hydrogen, alkali metal, ammonium, substituted ammonium, C₁-C₁₂-alkyl or polyoxyethylene.

As indicated above, the s-triazine derivative of formula (I) is obtained by reacting a cyanuric halide with same or different p-aminobenzoic acid esters. If the molar ratio of cyanuric halide to p-aminobenzoic acid ester is defined to be from 1:3 to 1:5 according to the invention, this molar ratio refers to the molar ratio of cyanuric halide relative to p-aminobenzoic acid ester, no matter whether same or different p-aminobenzoic acid esters are used in the reaction. In other words, it is to be understood that if the p-aminobenzoic acid esters reacted with the cyanuric halide are the same (i.e. R¹=R²=R³), the molar ratio of cyanuric acid to p-aminobenzoic acid ester is from 1:3 to 1:5, which means that three to five equivalents of one specific p-aminobenzoic acid ester are reacted with the cyanuric halide. On the other hand, if the p-aminobenzoic acid esters reacted with the cyanuric halide are different (e.g. R¹ 0 R² 0 R³), the molar ratio of cyanuric acid to p-aminobenzoic acid ester is also from 1:3 to 1:5, but the three to five equivalents of p-aminobenzoic acid ester relative to the cyanuric halide are represented by the total quantity of all different types of p-aminobenzoic acid esters.

Preferably, the p-aminobenzoic acid esters are purified by distillation prior to use.

As used herein, “alkali metals” include sodium, potassium, or lithium, preferably sodium or potassium. “Substituted ammonium” as used herein refers to ammonium in which one to four of the hydrogen atoms are independently replaced by C₁-C₆-alkyl, preferably C₁-C₂-alkyl.

The term “C₁-C₁₂ alkyl” as used herein denotes a straight-chain or branched alkyl group having from 1 to 12 carbon atoms. Preferred alkyl groups are C₆-C₁₀-alkyl, and particularly preferably 2-ethylhexyl.

“Polyoxyethylene” as used herein refers to a substituent of the structure H—(O—CH₂—CH₂)ₙ—OH, wherein n is from 1 to 1000, preferably from 1 to 100.

In the present invention, the process for preparing s-triazine derivatives further involves the purification of the product by gas/steam stripping. Generally, stripping is a physical separation process which uses a vapor stream to remove components from a liquid sample. As used herein, gas/steam stripping is performed by feeding a reaction product into a vessel or reactor and stripping off the non-polar solvent and optionally side products by adding water or a polar solvent or water steam or a steam of a polar solvent or a gas or a mixture thereof to the vessel or reactor. Thus, gas/steam stripping according to the invention also includes flashing or flash, which describes the process of adding water or a polar solvent in the liquid phase from the top part of the vessel/reactor. Preferably, gas/steam stripping is performed by adding water steam, a steam of a polar solvent, a gas, or mixtures thereof from the top or bottom part of the vessel/reactor. More preferably, gas/steam stripping is performed by adding water steam, a steam of a polar solvent, a gas, or mixtures thereof from the bottom part of the vessel/reactor. The overhead stream that comprises the desorbed solvent and optionally side products of the reaction can be condensed and separated. As used herein, the term “steam” in connection with gas/steam stripping refers to water steam or steam of a polar solvent, in particular steam of an alcohol, preferably steam of ethanol. As used herein the term “gas” in connection with gas/steam stripping refers to inert gas that does not undergo chemical reactions, in particular nitrogen gas. As described above, the term gas/steam stripping according to the invention also includes flashing or flash with water or a polar solvent, preferably ethanol, in the liquid phase.

As used herein, the pressure indicated by the pressure unit “bar” refers to relative pressure or gauge pressure, which is zero-referenced against ambient air pressure, so it is equal to absolute pressure minus atmospheric pressure. Gauge pressure may also be indicated by the pressure unit “barg”. Only in case the pressure unit “bar(abs)” is used, the pressure refers to absolute pressure.

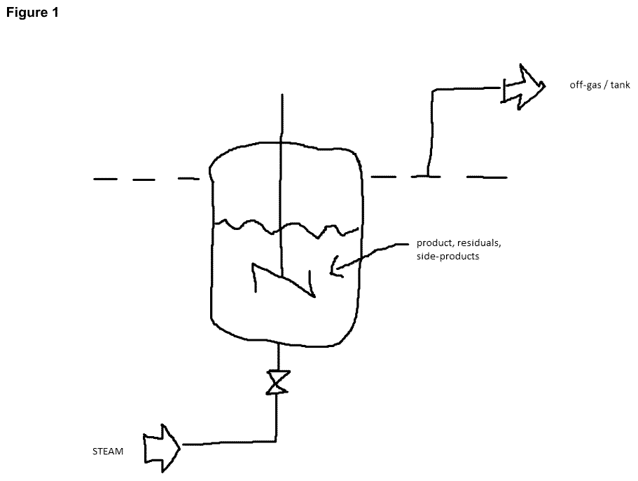

FIG. 1 illustrates how gas/steam stripping is preferably performed according to the invention, i.e. by feeding a reaction product into a vessel or reactor and stripping off the non-polar solvent and optionally side products by adding water steam, a steam of a polar solvent, a gas or a mixture thereof to the vessel or reactor, preferably from the bottom part of the vessel/reactor.

Preferred embodiments regarding the process of the invention are described hereinafter. The following general considerations apply to the process.

In general, the reaction steps are performed in reaction vessels customary for such reactions, e.g., conventional stirred tank reactors. The reactions may be carried out in a continuous or semi-batch-wise or batch-wise manner. Particularly preferably, the process of the invention is carried out in a semi-batch-wise manner, wherein at least one reactant is provided in a reaction vessel and at least one further reactant is added over a certain dosing time. In general, the process steps are preferably carried out under atmospheric pressure. Details regarding the reaction temperature are provided below. The end of the reaction can be monitored by methods known to a person skilled in the art, e.g., thin layer chromatography, GC, HPLC or NMR.

If not otherwise indicated, the reactants can in principle be contacted with one another in any desired sequence.

Furthermore, it is emphasized that the reaction steps may be performed on a technical scale.

In the following, preferred embodiments of the invention are provided. It is to be understood that the preferred embodiments of the invention are preferred alone or in combination with each other.

As indicated above, the present invention relates to a process for preparing an s-triazine derivative of formula (I), as provided above, comprising reacting in a non-polar solvent a cyanuric halide with p-aminobenzoic acid esters

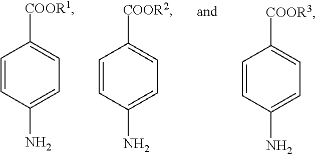

wherein R¹, R², and R³ are each same or different and independently selected from the group consisting of hydrogen, alkali metal, ammonium, substituted ammonium, C₁-C₁₂-alkyl or polyoxyethylene; and wherein the reaction product is purified by gas/steam stripping. In one embodiment, the molar ratio of cyanuric halide to p-aminobenzoic acid ester is from 1:2.9 to 1:5, preferably from 1:3 to 1:5. The excess of p-aminobenzoic acid ester relative to cyanuric halide is advantageous in order to achieve full conversion of the cyanuric halide, i.e. substitution of all three halides by same or different p-aminobenzoic acid esters. Residual p-aminobenzoic acid ester after the reaction can be removed by crystallization of the obtained s-triazine derivative in a polar solvent.

In one embodiment of the process of the invention, R²=R¹ and preferably R³=R²=R¹ and particularly preferably R¹, R², and R³ each represent a 2-ethylhexyl radical. Reacting p-aminobenzoic acid esters, wherein R¹, R², and R³ each represent a 2-ethylhexyl radical, gives rise to 2,4,6-trianilino-p-(carbo-2′-ethylhexyl-1′-oxy)-1,3,5-triazine as product, which represents the known highly effective UV absorber ethylhexyl triazone (Uvinul T 150, CAS number: 88122-99-0).

Therefore, in one embodiment of the process of the invention, the s-triazine derivative is 2,4,6-trianilino-p-(carbo-2′-ethylhexyl-1′-oxy)-1,3,5-triazine having the following chemical formula (Ia):

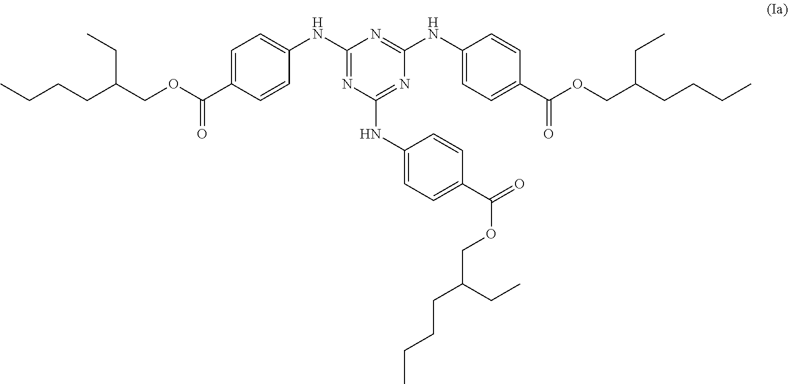 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

As indicated above, gas/steam stripping is performed for purification of the product s-triazine derivative of formula (I).

In one embodiment of the process of the invention, gas/steam stripping is performed by feeding the reaction product into a vessel or reactor and stripping off the non-polar solvent by adding water or a polar solvent or water steam or a steam of a polar solvent or a gas or a mixture thereof to the vessel or reactor from the top or bottom part of the vessel/reactor. Preferably, water steam or a steam of a polar solvent or a gas or a mixture thereof is added from the bottom part of the vessel/reactor. If stripping is performed with a steam of a polar solvent, preferably ethanol steam is used. If stripping is performed with a gas, preferably nitrogen gas is used. Thus, in one embodiment, gas/steam stripping is performed by adding water steam, ethanol steam, or nitrogen gas, preferably water steam or nitrogen gas, preferably from the bottom part of the vessel/reactor. The overhead stream that comprises the desorbed solvent and optionally side products of the reaction can be condensed and separated. Thus, gas/steam stripping is advantageous to effectively remove solvent residues and side products from the desired s-triazine derivative. The resulting s-triazine derivative is obtained in high yield and high purity. Moreover, gas/steam stripping has the advantage of being applicable on a large industrial scale. Further, it has been found that the process provides the advantage of a reaction product with optimum physical properties in terms of solubility and stability in oils used for cosmetic formulations. As described above, the solubility in said oils is decreased if too high temperatures are used for the solvent removal.

In one embodiment of the process of the invention, the temperature during gas/steam stripping is less than 200 °C, preferably less than 180 °C. In another embodiment, the temperature during gas/steam stripping is from 100 °C to 200 °C, preferably from 100 °C to 180 °C. In another embodiment, the temperature during gas/steam stripping is from 120 °C to 170 °C. In another embodiment, the temperature during gas/steam stripping is from 140 °C to 160 °C.

In one embodiment, gas/steam stripping is performed for a time period of up to 48 hours. In another embodiment, gas/steam stripping is performed for a time period of from 1 to 24 hours. In another embodiment, gas/steam stripping is performed for a time period of from 2 to 12 hours. In another embodiment, gas/steam stripping is performed for a time period of from 4 to 7 hours.

In one embodiment of the invention, the pressure of the gas/steam during gas/steam stripping is up to 100 bar, preferably up to 20 bar. In another embodiment, the pressure of the gas/steam during gas/steam stripping is from 0.1 bar to 20 bar. In another embodiment, the pressure of the gas/steam during gas/steam stripping is from 0.5 bar to 10 bar. In another embodiment, the pressure of the gas/steam during gas/steam stripping is from 1 bar to 6 bar. In one embodiment, the temperature during steam/gas stripping is less than 200 °C, preferably less than 180 °C; and the pressure of the steam/gas during steam/gas stripping is up to 100 bar, preferably up to 20 bar. In one embodiment, the temperature during gas/steam stripping is from 120 °C to 170 °C and the pressure of the gas/steam during gas/steam stripping is from 0.1 bar to 20 bar. In one embodiment, the temperature during gas/steam stripping is from 120 °C to 170 °C, and the pressure of the gas/steam during gas/steam stripping is from 0.5 bar to 10 bar. In another embodiment, the temperature during gas/steam stripping is from 140 °C to 160 °C and the pressure of the gas/steam during gas/steam stripping is from 1 bar to 6 bar. In one embodiment, the temperature during gas/steam stripping is from 140 °C to 160 °C and the pressure of the gas/steam during gas/steam stripping is from 0.1 bar to 20 bar. In another embodiment, the temperature during gas/steam stripping is from 140 °C to 160 °C and the pressure of the gas/steam during gas/steam stripping is from 0.5 bar to 10 bar. In another embodiment, the temperature during gas/steam stripping is from 140 °C to 160 °C and the pressure of the gas/steam during gas/steam stripping is from 1 bar to 6 bar. In one particular embodiment, gas/steam stripping is performed at a temperature of from 120 °C to 170 °C and a pressure of from 0.5 bar to 10 bar for a time period of from 1 to 12 hours. In another particular embodiment, gas/steam stripping is performed at a temperature of from 120 °C to 170 °C and a pressure of from 0.5 bar to 10 bar for a time period of from 3 to 7 hours.

In one embodiment of the process of the invention, the amount of gas/steam used for gas/steam stripping is at least 25% in weight relative to the crude product. In another embodiment, the amount of gas/steam used for gas/steam stripping is at least 50% in weight relative to the crude product.

In one embodiment of the process of the invention, the non-polar solvent and water are azeotropically distilled prior to gas/steam stripping. In one embodiment, the temperature during azeotropic distillation is less than 200 °C, preferably less than 180 °C. In another embodiment, the temperature during azeotropic distillation is from 140 °C to 180 °C. In one embodiment, the temperature during azeotropic distillation is from 140 °C to 180 °C and the pressure is from 1 mbar(abs) to 2 bar(abs). In another embodiment, the temperature during azeotropic distillation is from 140 °C to 180 °C and the pressure is from 10 mbar(abs) to 1 bar(abs). In another embodiment, the temperature during azeotropic distillation is from 140 °C to 180 °C and the pressure is from 20 mbar(abs) to 100 mbar(abs).

In one embodiment, the s-triazine derivative is dissolved in a polar solvent after gas/steam stripping for subsequent crystallization of the product.

In one embodiment of the process of the invention, the s-triazine derivative is dissolved in a polar solvent, preferably a C₁-C₈-alcohol or water or a mixture thereof. The C₁-C₈-alcohol includes straight-chain, branched, or cyclic alcohols having from 1 to 8 carbon atoms or mixtures thereof, preferably ethanol, n-propanol, isopropanol, n-butanol, tert-butanol, n-pentanol, n-hexanol, n-octanol, 2-ethylhexanol, and cyclohexanol, and especially preferably ethanol. Thus, in another embodiment of the process of the invention, the s-triazine derivative is dissolved in a, optionally aliphatic, C₂-C₈-alcohol or water or a mixture thereof. In another embodiment, the s-triazine derivative is dissolved in a mixture of at least one C₁-C₄-alcohol with up to 10% by weight of water, preferably in a mixture of at least one C₂-C₄-alcohol with up to 10% by weight of water. In another embodiment, the s-triazine derivative is dissolved in a mixture of ethanol and at least one C₃- or C₄-alcohol with up to 10% by weight of water. In another embodiment, the s-triazine derivative is dissolved in ethanol with up to 10% by weight of water. The water content may be lower, for example up to 5% by weight, up to 3% by weight or even less. In some embodiments, the water content may be lower than 1% by weight. In various embodiments, the s-triazine derivative is washed with pure ethanol.

If the solvent or mixture contains ethanol, then common denaturants can be used additionally, for example butan-2-one, 3-methylbutan-2-one, 5-methylheptan-3-one, isopropanol, tert-butanol, petrolether, toluene, cyclohexane, 2-methylpropan-2-ol. Thus, in one embodiment, the at least one C₁-C₄-alcohol, at least one C₂-C₄-alcohol, at least one C₃- or C₄-alcohol only refers to the denaturant added for denaturation of ethanol. Adding the polar solvent decreases the temperature of the vessel/reactor, which provides avoidance of hydrolysis of the esters of the s-triazine derivative or transesterification of the s-triazine derivative. Thus, in one embodiment, the temperature of the vessel/reactor is decreased prior to the crystallization. In one particular embodiment, the temperature is already decreased to some extent prior to the addition of the polar solvent by flashing with water. As a by-effect, flashing is further removing solvent residues and optionally impurities. In one embodiment, the dissolution of the s-triazine derivative in the polar solvent is followed by filtration of the solution, crystallization of the s-triazine derivative, washing with a polar solvent and drying. The polar solvent is preferably an alcohol as described above. In various embodiments, the process comprises crystallization in the presence of one or more alcohol solvents as described above for producing the tautomeric form I of the compound of formula (Ia).

As described above, the reaction of the cyanuric halide with p-aminobenzoic acid esters is performed in a non-polar solvent. In one embodiment of the process of the invention, the reaction is performed in an aromatic solvent, which is preferably toluene, xylene, a xylene isomeric mixture, trimethylbenzenes, a trimethylbenzene isomeric mixture, or a mixture thereof. In a preferred embodiment, the solvent is azeotropically dewatered prior to the start of the reaction. Removal of residual water is advantageous in terms of high yield and low number of side products, as cyanuric halides have the tendency to hydrolyze giving rise to mono-, di-, and/or trihydroxy-triazines. In addition, under the elevated process temperatures during s-triazine derivative formation, partial or full hydrolysis and/or reesterification of the p-aminobenzoic acid esters, intermediates, and/or the desired s-triazine derivative can occur. In one particular embodiment, the water content is less than 300 mg/kg.

In another embodiment of the process of the invention, the cyanuric halide is introduced as initial charge as suspension with the dewatered non-polar solvent. In a particular embodiment, the vessel/reactor is precharged with the solvent, preferably at an elevated temperature and more preferably at a temperature of 50 °C to 90 °C. This is advantageous in order to prevent (partial) hydrolysis of the cyanuric halide at too high temperatures.

In another embodiment of the process of the invention, the p-aminobenzoic acid ester is added to the dewatered non-polar solvent in a separate vessel/reactor. Preferably, the p-aminobenzoic acid ester is purified by vacuum distillation beforehand. In one embodiment, the cyanuric halide suspension is then dosed to the p-aminobenzoic acid ester in dewatered non-polar solvent to start the reaction. In a particular embodiment, additional dewatered non-polar solvent is added.

In one embodiment of the process of the invention, the reaction is performed at a temperature of from 75 °C to the boiling point of the used solvent for a time period of up to 48 h. In another embodiment, the reaction is performed at a temperature of from 75 °C to the boiling point of the used solvent for a time period of from 0.5 h to 24 h. In another embodiment, the reaction is performed at a temperature of from 75 °C to the boiling point of the used solvent for a time period of from 0.5 h to 12 h. In another embodiment, the reaction is performed at a temperature of from 75 °C to the boiling point of the used solvent for a time period of from 1 h to 3 h. In another embodiment, the reaction is performed at a temperature of from 100 °C to 150 °C for a time period of from 0.5 h to 12 h. In another embodiment, the reaction is performed at a temperature of from 100 °C to 150 °C for a time period of from 1 h to 3 h. In another embodiment, the reaction is performed at a temperature of from 110 °C to 130 °C for a time period of from 0.5 h to 12 h. In another embodiment, the reaction is performed at a temperature of from 110 °C to 130 °C for a time period of from 1 h to 3 h. Performing the reaction for a longer reaction time does not negatively affect the product yield and purity but reduces the space-time-yield of the process and thus increases waste and costs.

In another embodiment, a base is added after the reaction in order to neutralize the reaction mixture. Preferably, the base is selected from ammonia, alkali metal hydroxide, alkali metal carbonate, or alkali metal bicarbonate. In one embodiment, the base is selected from alkali metal carbonate, preferably potassium carbonate or sodium carbonate. In another embodiment, the base is sodium carbonate.

In one embodiment of the process of the invention, the cyanuric halide is selected from cyanuric chloride, cyanuric bromide and mixtures thereof. Preferably, the cyanuric halide is cyanuric chloride.

As described above, the s-triazine derivative of formula (I) is prepared by reacting a cyanuric halide with same or different p-aminobenzoic acid esters. In one embodiment of the process of this invention, the process further comprises preparing p-aminobenzoic acid esters comprising reacting p-nitrobenzoic acid esters with hydrogen in the presence of a catalyst; preferably preparing p-aminobenzoic acid-2-ethyl hexylester comprising reacting p-nitrobenzoic acid-2-ethyl hexylester with hydrogen in the presence of a catalyst. In another embodiment, the process further comprises preparing p-nitrobenzoic acid esters comprising reacting p-nitrobenzoic acid with an alcohol or polyoxyethylene; preferably preparing p-nitrobenzoic acid-2-ethyl hexylester comprising reacting p-nitrobenzoic acid with 2-ethylhexanol.

As described above, high purity of the s-triazine derivative can be obtained by the process of this invention by purification of the product with gas/steam stripping. Thus, in one embodiment the present invention relates to an s-triazine derivative of formula (I) having a purity of at least 98% by weight. In another embodiment, the present invention relates to an s-triazine derivative of formula (I) having a purity of at least 99% by weight. In another embodiment, the present invention relates to an s-triazine derivative of formula (I) having a purity of at least 99.5% by weight.

In addition, the s-triazine derivative obtained by this process has optimum physical properties, e.g., in terms of particle size. Therefore, in one embodiment, the present invention relates to an s-triazine derivative, which is in the form of pourable or flowable particles, preferably in crystalline form, particularly preferably with an average particle diameter of from 5 μm up to 500 μm, and/or a bulk density of greater than 0.2 g/mL. In another embodiment, the present invention relates to an s-triazine derivative with an average particle diameter of from 5 μm up to 500 μm, and/or a bulk density of from 0.3 g/mL to 1.5 g/mL. In another embodiment, the present invention relates to an s-triazine derivative with an average particle diameter of from 5 μm up to 500 μm, and/or a bulk density of from 0.4 g/mL to 0.8 g/mL. In another embodiment, the present invention relates to an s-triazine derivative with an average particle diameter of from 20 μm up to 200 μm, and/or a bulk density of from 0.4 g/mL to 0.8 g/mL. In another embodiment, the present invention relates to an s-triazine derivative with an average particle diameter of from 30 μm up to 100 μm, and/or a bulk density of from 0.4 g/mL to 0.8 g/mL. An s-triazine derivative with these properties is advantageous, e.g., in terms of high solubility and stability in the oils used in cosmetic formulations. Further, the flowability and particle size provide the advantage of easy handling and dosing.

Thus, in one embodiment, the present invention relates to an s-triazine derivative with the above properties, which is obtainable by the process of this invention. As described above, s-triazine derivatives are known for their UV absorption efficiency in both UVB and UVA regions with high light stability. In one embodiment, the present invention relates to the use of the s-triazine derivative of formula (I) as a light protecting agent. Considering the high purity and physical properties of the s-triazine derivative obtained by the process of this invention, its use as light protecting agent is advantageous for, e.g., cosmetic preparations.

Thus, in one embodiment, the present invention relates to a cosmetic preparation comprising the s-triazine derivative of formula (I). In particular, in one embodiment, the present invention relates to a cosmetic preparation comprising the s-triazine derivative of formula (I) in the form of pourable or flowable particles, preferably in crystalline form, and particularly preferably with an average particle diameter and bulk density as described above.

In a further aspect and embodiment, the present invention relates to a process for preparing an s-triazine derivative, wherein the s-triazine derivative is 2,4,6-trianilino-p-(carbo-2′-ethylhexyl-1′-oxy)-1,3,5-triazine having the following chemical formula (Ia):

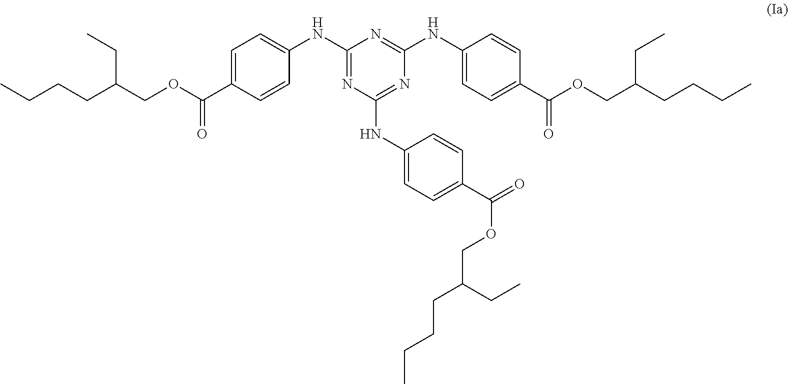 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

comprising reacting in a non-polar solvent a cyanuric halide with p-aminobenzoic acid-2-ethyl hexylesters

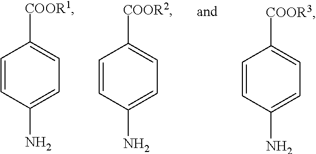

wherein R¹, R², and R³ each represent a 2-ethylhexyl radical; and wherein the reaction product is purified by gas/steam stripping. In one embodiment, the molar ratio of cyanuric halide to p-aminobenzoic acid ester is from 1:2.9 to 1:5, preferably from 1:3 to 1:5. The excess of p-aminobenzoic acid ester relative to cyanuric halide is advantageous in order to achieve full conversion of the cyanuric halide, i.e. substitution of all three halides by same or different p-aminobenzoic acid esters. Residual p-aminobenzoic acid ester after the reaction can be removed by crystallization of the obtained s-triazine derivative in a polar solvent.

As indicated above, gas/steam stripping is performed for purification of the product s-triazine derivative of formula (Ia).

In one embodiment of the process of the invention, gas/steam stripping is performed by feeding the reaction product into a vessel or reactor and stripping off the non-polar solvent by adding water or a polar solvent or water steam or a steam of a polar solvent or a gas or a mixture thereof to the vessel or reactor from the top or bottom part of the vessel/reactor. Preferably, water steam or a steam of a polar solvent or a gas or a mixture thereof is added from the bottom part of the vessel/reactor. If stripping is performed with a steam of a polar solvent, preferably ethanol steam is used. If stripping is performed with a gas, preferably nitrogen gas is used. Thus, in one embodiment, gas/steam stripping is performed by adding water steam, ethanol steam, or nitrogen gas, preferably water steam or nitrogen gas, preferably from the bottom part of the vessel/reactor. The overhead stream that comprises the desorbed solvent and optionally side products of the reaction can be condensed and separated. Thus, gas/steam stripping is advantageous to effectively remove solvent residues and side products from the desired s-triazine derivative. The resulting s-triazine derivative is obtained in high yield and high purity. Moreover, gas/steam stripping has the advantage of being applicable on a large industrial scale. Further, it has been found that the process provides the advantage of a reaction product with optimum physical properties in terms of solubility and stability in oils used for cosmetic formulations. As described above, the solubility in said oils is decreased if too high temperatures are used for the solvent removal.

In one embodiment of the process of the invention, the temperature during gas/steam stripping is less than 200 °C, preferably less than 180 °C. In another embodiment, the temperature during gas/steam stripping is from 100 °C to 200 °C, preferably from 100 °C to 180 °C. In another embodiment, the temperature during gas/steam stripping is from 120 °C to 170 °C. In another embodiment, the temperature during gas/steam stripping is from 140 °C to 160 °C.

In one embodiment, gas/steam stripping is performed for a time period of up to 48 hours. In another embodiment, gas/steam stripping is performed for a time period of from 1 to 24 hours. In another embodiment, gas/steam stripping is performed for a time period of from 2 to 12 hours. In another embodiment, gas/steam stripping is performed for a time period of from 4 to 7 hours.

In one embodiment of the invention, the pressure of the gas/steam during gas/steam stripping is up to 100 bar, preferably up to 20 bar. In another embodiment, the pressure of the gas/steam during gas/steam stripping is from 0.1 bar to 20 bar. In another embodiment, the pressure of the gas/steam during gas/steam stripping is from 0.5 bar to 10 bar. In another embodiment, the pressure of the gas/steam during gas/steam stripping is from 1 bar to 6 bar. In one embodiment, the temperature during steam/gas stripping is less than 200 °C, preferably less than 180 °C; and the pressure of the steam/gas during steam/gas stripping is up to 100 bar, preferably up to 20 bar. In one embodiment, the temperature during gas/steam stripping is from 120 °C to 170 °C and the pressure of the gas/steam during gas/steam stripping is from 0.1 bar to 20 bar. In one embodiment, the temperature during gas/steam stripping is from 120 °C to 170 °C, and the pressure of the gas/steam during gas/steam stripping is from 0.5 bar to 10 bar. In another embodiment, the temperature during gas/steam stripping is from 140 °C to 160 °C and the pressure of the gas/steam during gas/steam stripping is from 1 bar to 6 bar. In one embodiment, the temperature during gas/steam stripping is from 140 °C to 160 °C and the pressure of the gas/steam during gas/steam stripping is from 0.1 bar to 20 bar. In another embodiment, the temperature during gas/steam stripping is from 140 °C to 160 °C and the pressure of the gas/steam during gas/steam stripping is from 0.5 bar to 10 bar. In another embodiment, the temperature during gas/steam stripping is from 140 °C to 160 °C and the pressure of the gas/steam during gas/steam stripping is from 1 bar to 6 bar. In one particular embodiment, gas/steam stripping is performed at a temperature of from 120 °C to 170 °C and a pressure of from 0.5 bar to 10 bar for a time period of from 1 to 12 hours. In another particular embodiment, gas/steam stripping is performed at a temperature of from 120 °C to 170 °C and a pressure of from 0.5 bar to 10 bar for a time period of from 3 to 7 hours.

In one embodiment of the process of the invention, the amount of gas/steam used for gas/steam stripping is at least 25% in weight relative to the crude product. In another embodiment, the amount of gas/steam used for gas/steam stripping is at least 50% in weight relative to the crude product.

In one embodiment of the process of the invention, the non-polar solvent and water are azeotropically distilled prior to gas/steam stripping. In one embodiment, the temperature during azeotropic distillation is less than 200 °C, preferably less than 180 °C. In another embodiment, the temperature during azeotropic distillation is from 140 °C to 180 °C. In one embodiment, the temperature during azeotropic distillation is from 140 °C to 180 °C and the pressure is from 1 mbar(abs) to 2 bar(abs). In another embodiment, the temperature during azeotropic distillation is from 140 °C to 180 °C and the pressure is from 10 mbar(abs) to 1 bar(abs). In another embodiment, the temperature during azeotropic distillation is from 140 °C to 180 °C and the pressure is from 20 mbar(abs) to 100 mbar(abs).

In one embodiment, the s-triazine derivative is dissolved in a polar solvent after gas/steam stripping for subsequent crystallization of the product. Said solvent may be an alcohol as described herein above.

In one embodiment of the process of the invention, the s-triazine derivative is dissolved in a polar solvent, preferably a C₁-C₈-alcohol or water or a mixture thereof. The C₁-C₈-alcohol includes straight-chain, branched, or cyclic alcohols having from 1 to 8 carbon atoms or mixtures thereof, preferably ethanol, n-propanol, isopropanol, n-butanol, tert-butanol, n-pentanol, n-hexanol, n-octanol, 2-ethylhexanol, and cyclohexanol, and especially preferably ethanol. Thus, in another embodiment of the process of the invention, the s-triazine derivative is dissolved in a C₂-C₈-alcohol or water or a mixture thereof. In another embodiment, the s-triazine derivative is dissolved in a mixture of at least one C₁-C₄-alcohol with up to 10% by weight of water, preferably in a mixture of at least one C₂-C₄-alcohol with up to 10% by weight of water. In another embodiment, the s-triazine derivative is dissolved in a mixture of ethanol and at least one C₃- or C₄-alcohol with up to 10% by weight of water. Thus, in another embodiment, the s-triazine derivative is dissolved in ethanol with up to 10% by weight of water. If the solvent or mixture contains ethanol, then common denaturants can be used additionally, for example butan-2-one, 3-methylbutan-2-one, 5-methylheptan-3-one, isopropyl alcohol, petrolether, toluene, cyclohexane, 2-methylpropan-2-ol. Thus, in one embodiment, the at least one C₁-C₄-alcohol, at least one C₂-C₄-alcohol, at least one C₃- or C₄-alcohol only refers to the denaturant added for denaturation of ethanol. Adding the polar solvent decreases the temperature of the vessel/reactor, which provides avoidance of hydrolysis of the esters of the s-triazine derivative or transesterification of the s-triazine derivative. Thus, in one embodiment, the temperature of the vessel/reactor is decreased prior to the crystallization. In one particular embodiment, the temperature is already decreased to some extent prior to the addition of the polar solvent by flashing with water. As a by-effect, flashing is further removing solvent residues and optionally impurities. In one embodiment, the dissolution of the s-triazine derivative in the polar solvent is followed by filtration of the solution, crystallization of the s-triazine derivative, washing with a polar solvent and drying.

As described above, the reaction of the cyanuric halide with p-aminobenzoic acid esters is performed in a non-polar solvent. In one embodiment of the process of the invention, the reaction is performed in an aromatic solvent, which is preferably toluene, xylene, a xylene isomeric mixture, trimethylbenzenes, a trimethylbenzene isomeric mixture, or a mixture thereof. In a preferred embodiment, the solvent is azeotropically dewatered prior to the start of the reaction. Removal of residual water is advantageous in terms of high yield and low number of side products, as cyanuric halides have the tendency to hydrolyze giving rise to mono-, di-, and/or trihydroxy-triazines. In addition, under the elevated process temperatures during s-triazine derivative formation, partial or full hydrolysis and/or reesterification of the p-aminobenzoic acid esters, intermediates, and/or the desired s-triazine derivative can occur. In one particular embodiment, the water content is less than 300 mg/kg.

In another embodiment of the process of the invention, the cyanuric halide is introduced as initial charge as suspension with the dewatered non-polar solvent. In a particular embodiment, the vessel/reactor is precharged with the solvent, preferably at an elevated temperature and more preferably at a temperature of 50 °C to 90 °C. This is advantageous in order to prevent (partial) hydrolysis of the cyanuric halide at too high temperatures.

In another embodiment of the process of the invention, the p-aminobenzoic acid-2-ethyl hexylester is added to the dewatered non-polar solvent in a separate vessel/reactor. Preferably, the p-aminobenzoic acid-2-ethyl hexylester is purified by vacuum distillation beforehand. In one embodiment, the cyanuric halide suspension is then dosed to the p-aminobenzoic acid-2-ethyl hexylester in dewatered non-polar solvent to start the reaction. In a particular embodiment, additional dewatered non-polar solvent is added.

In one embodiment of the process of the invention, the reaction is performed at a temperature of from 75 °C to the boiling point of the used solvent for a time period of up to 48 h. In another embodiment, the reaction is performed at a temperature of from 75 °C to the boiling point of the used solvent for a time period of from 0.5 h to 24 h. In another embodiment, the reaction is performed at a temperature of from 75 °C to the boiling point of the used solvent for a time period of from 0.5 h to 12 h. In another embodiment, the reaction is performed at a temperature of from 75 °C to the boiling point of the used solvent for a time period of from 1 h to 3 h. In another embodiment, the reaction is performed at a temperature of from 100 °C to 150 °C for a time period of from 0.5 h to 12 h. In another embodiment, the reaction is performed at a temperature of from 100 °C to 150 °C for a time period of from 1 h to 3 h. In another embodiment, the reaction is performed at a temperature of from 110 °C to 130 °C for a time period of from 0.5 h to 12 h. In another embodiment, the reaction is performed at a temperature of from 110 °C to 130 °C for a time period of from 1 h to 3 h. Performing the reaction for a longer reaction time does not negatively affect the product yield and purity but reduces the space-time-yield of the process and thus increases waste and costs.

In another embodiment, a base is added after the reaction in order to neutralize the reaction mixture. Preferably, the base is selected from ammonia, alkali metal hydroxide, alkali metal carbonate, or alkali metal bicarbonate. In one embodiment, the base is selected from alkali metal carbonate, preferably potassium carbonate or sodium carbonate. In another embodiment, the base is sodium carbonate.

In one embodiment of the process of the invention, the cyanuric halide is selected from cyanuric chloride, cyanuric bromide and mixtures thereof. Preferably, the cyanuric halide is cyanuric chloride.

As described above, the s-triazine derivative of formula (Ia) is prepared by reacting a cyanuric halide with p-aminobenzoic acid-2-ethyl hexylester. In one embodiment of the process of this invention, the process further comprises preparing p-aminobenzoic acid-2-ethyl hexylester comprising reacting p-nitrobenzoic acid-2-ethyl hexylester with hydrogen in the presence of a catalyst. In another embodiment, the process further comprises preparing p-nitrobenzoic acid-2-ethyl hexylester comprising reacting p-nitrobenzoic acid with 2-ethylhexanol.

As described above, high purity of the s-triazine derivative can be obtained by the process of this invention by purification of the product with gas/steam stripping. Thus, in one embodiment the present invention relates to an s-triazine derivative of formula (Ia) having a purity of at least 98% by weight. In another embodiment, the present invention relates to an s-triazine derivative of formula (Ia) having a purity of at least 99% by weight. In another embodiment, the present invention relates to an s-triazine derivative of formula (Ia) having a purity of at least 99.5% by weight. In even more preferred embodiments, the purity may be at least 99.6, 99.7, 99.8 or 99.9% by weight. It may be preferred that the content of a given impurity, such as any one of p-aminobenzoic acid (AB), mono hydroxide of ethyl hexyl triazone (MHT), mono carboxylic acid of ethyl hexyl triazone (MCS), and mono ethylester of ethyl hexyl triazone (MEE), in the final product is below 0.2% by weight, preferably 0.1% by weight or lower, more preferably 0.05% by weight or lower. In preferred embodiments, the concentration of AB and/or MCS and/or MHT is 0.2% by weight or lower, preferably 0.1% by weight or lower, more preferably 0.05% by weight or lower. In a preferred embodiment, the concentration of AB is 0.2% by weight or lower, preferably 0.1% by weight or lower, more preferably 0.05% by weight or lower. These values refer to concentrations as measured by HPLC. Further, optionally in addition to the low concentrations of the other impurities, the residual content of solvent, such as xylene, may be 100 ppm or lower, preferably 10 ppm or lower, as determined by GC.

In various embodiments, the s-triazine derivative is tautomeric form I of 2,4,6-trianilino-p-(carbo-2′ethylhexyl-1′-oxy)-1,3,5-triazine. Said tautomeric form may be obtained by the synthesis as described in WO 03/074499 A1 or as described in example 1 below and then subjected to the further purification steps as described herein. The present invention thus also relates to tautomeric form I of 2,4,6-trianilino-p-(carbo-2′ethylhexyl-1′-oxy)-1,3,5-triazine, which is obtainable by the process of the present invention.

In various embodiments, the tautomeric purity of the product, i.e. of 2,4,6-trianilino-p-(carbo-2′ethylhexyl-1′-oxy)-1,3,5-triazine, as determined by IR spectroscopy is such that there is no double band for the carbonyl groups at 1694 and 1717 cm⁻¹, but only a single band at 1697 cm⁻¹. It may also be preferred that the single band at about 1697 cm⁻¹ does not have a shoulder indicating the presence of certain amounts of undesired tautomeric form(s) of 2,4,6-trianilino-p-(carbo-2′ethylhexyl-1′-oxy)-1,3,5-triazine. It has been found that the tautomeric purity of the product of the synthesis described in example 1 below and also in WO 03/074499 A1 is not altered by the following additional purification steps of steam/gas stripping.

In addition, the s-triazine derivative obtained by this process has optimum physical properties, e.g., in terms of particle size. Therefore, in one embodiment, the present invention relates to an s-triazine derivative, which is in the form of pourable or flowable particles, preferably in crystalline form, particularly preferably with an average particle diameter of from 5 μm up to 500 μm, and/or a bulk density of greater than 0.2 g/mL. In another embodiment, the present invention relates to an s-triazine derivative with an average particle diameter of from 5 μm up to 500 μm, and/or a bulk density of from 0.3 g/mL to 1.5 g/mL. In another embodiment, the present invention relates to an s-triazine derivative with an average particle diameter of from 5 μm up to 500 μm, and/or a bulk density of from 0.4 g/mL to 0.8 g/mL. In another embodiment, the present invention relates to an s-triazine derivative with an average particle diameter of from 20 μm up to 200 μm, and/or a bulk density of from 0.4 g/mL to 0.8 g/mL. In another embodiment, the present invention relates to an s-triazine derivative with an average particle diameter of from 30 μm up to 100 μm, and/or a bulk density of from 0.4 g/mL to 0.8 g/mL. An s-triazine derivative with these properties is advantageous, e.g., in terms of high solubility and stability in the oils used in cosmetic formulations. Further, the flowability and particle size provide the advantage of easy handling and dosing.

Thus, in one embodiment, the present invention relates to an s-triazine derivative with the above properties, which is obtainable by the process of this invention. As described above, s-triazine derivatives are known for their UV absorption efficiency in both UVB and UVA regions with high light stability. In one embodiment, the present invention relates to the use of the s-triazine derivative of formula (Ia) as a light protecting agent. Considering the high purity and physical properties of the s-triazine derivative obtained by the process of this invention, its use as light protecting agent is advantageous for, e.g., cosmetic preparations.

Thus, in one embodiment, the present invention relates to a cosmetic preparation comprising the s-triazine derivative of formula (Ia). In particular, in one embodiment, the present invention relates to a cosmetic preparation comprising the s-triazine derivative of formula (Ia) in the form of pourable or flowable particles, preferably in crystalline form, and particularly preferably with an average particle diameter and bulk density as described above.

The present invention is further illustrated by the following examples.

### EXAMPLES

The following abbreviations are used: AB: p-aminobenzoic acid, ABE: 2-ethylhexyl p-aminobenzoate, MHT: mono hydroxide of ethyl hexyl triazone, MCS: mono carboxylic acid of ethyl hexyl triazone, MEE: mono ethylester of ethyl hexyl triazone, MCT: mono chloride of ethyl hexyl triazone, EHT: ethyl hexyl triazone.

In the example, GC analysis is performed on an Agilent Technologies 7890B using the following conditions and parameters:
Detector: FID
Solvent: Dichloromethane
Column: HP-5 by Agilent Technologies (30 m, 0.32 mm ID, 0.25 μm film thickness)
Carrier Gas:Nitrogen 1 mL/min (Split 100:1)
Injector temperature: 270 °C
Detector temperature: 300 °C
Temperature program: 80 °C for 3 min, followed by 50 °C/min until 130 °C and keeping 130 °C for 4 min.

In the example, HPLC analysis is performed on an Agilent Technologies 1260 using the following conditions and parameters:
Detector: UV at 295 nm
Column: PerfectBond C8-HD by MZ Analysentechnik (5 μm, 125×4 mm)
Flow: 0.7 mL/min
Solvent: THE (Solvent A) and aqueous 0.1% H₂SO₄ (pH 1.5) (Solvent B)

### Gradient Program:

| Time [min] | Solvent A [vol %] | Solvent B [vol %] |
|---|---|---|
| 0.00 | 40.0 | 60.0 |
| 15.00 | 70.0 | 30.0 |
| 25.00 | 70.0 | 30.0 |
| 30.00 | 40.0 | 60.0 |
| 35.00 | 40.0 | 60.0 |

### Example 1: Preparation of ethyl hexyl triazone (Uvinul T 150)

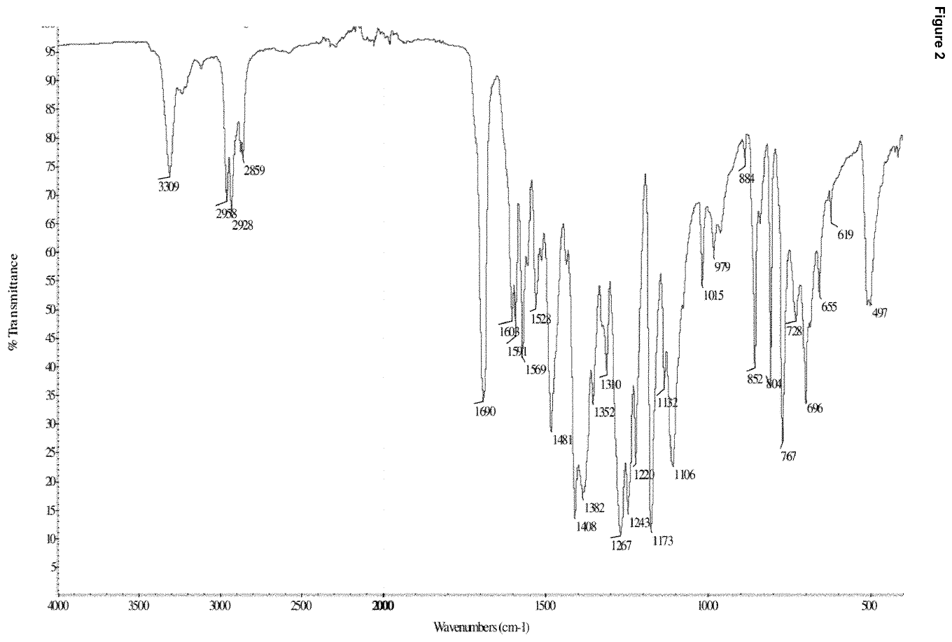

378 kg of xylene was refluxed in reactor 1 until the water content was less than 250 mg/kg. 180 kg of the as-purified xylene were purged into a separate reactor 2. The transfer preferably took place at temperatures of 60-80 °C. Afterwards, 100 kg of cyanuric chloride were added to reactor 2 and stirred to receive a cyanuric chloride suspension in xylene at an elevated temperature of less than 100 °C. Afterwards, 430 kg of purified 2-ethylhexyl p-aminobenzoate, which had been purified beforehand by distillation, were dosed to the remaining purified xylene in reactor 1. Afterwards, the suspension in reactor 2 and additional 110 l of pure xylene were dosed to reactor 1 to start the reaction. During the reaction, the reactor 1 was heated up to 122 °C. After 2 hours, 175 kg of 20 weight % Na₂CO₃ in water was added in 4 steps to reactor 1 to achieve a pH-value of 6.5 to 8.0. Afterwards, the content of reactor 1 was transferred to reactor 3. Afterwards, xylene and water were azeotropically distilled at 155 °C and a pressure of 30 mbar(abs) for 6 hours. Afterwards, the temperature was set to 160 °C. At that step the xylene content was 5500 ppm (determined by GC). Afterwards, the remaining evanescent residuals (mainly water and xylene) were stripped by adding water steam with a pressure of 5.5 bar(abs) and a temperature of 155 °C and a flow rate of 50 kg/h for 5 hours. Afterwards, the temperature of reactor 3 was decreased by flashing with 11 l water. Further temperature decrease was achieved by adding 790 kg of purified denatured ethanol (e.g. 100 liter of ethanol is denatured by adding 78 g of tert-butanol and 5 kg of iso propanol) to achieve an ethanolic solution of ethyl hexyl triazone. Afterwards, the solution of ethyl hexyl triazone was filtered, crystallized and washed with additional ethanol followed by drying to achieve a pure powder of ethyl hexyl triazone with a yield of 420 kg. Characterization of the final product by HPLC is provided in Table 1 and by IR spectroscopy in FIG. 2. The IR spectroscopy shows that the product obtained has high tautomeric purity (Tautomer I), as there is no peak or shoulder in the range of 1700 to 1720 cm⁻¹, but only a single peak at 1690 cm⁻¹. The content of xylene measured by GC is <10 ppm.

TABLE 1

| | Yield EHT | EHT | AB/ | MHT/ | MCS/ | MEE/ | MCT |
|---|---|---|---|---|---|---|---|
| Example | kg | area % | weight % | weight % | weight % | weight % | weight % |
| 1 | 420 | 99.80 | 0.1 | 0.04 | 0.02 | 0.01 | 0.02 |

### Examples 2 to 6: Purification by Steam Stripping

After following a similar reaction procedure as described in Example 1, 1254 g of the reaction product of reactor 1 was transferred to reactor 3 and azeotropically distilled. Afterwards, the remaining evanescent residuals were stripped by adding water steam with a temperature of 155 °C, a pressure p and a flow rate f for a time period t. Afterwards, the sample was filtered, washed with ethanol and dried. Characterization by HPLC (AB, MHT, MCS, MEE) and GC (xylene) is provided in Table 2.

TABLE 2

| | | | | Xylene | Xylene | | | | | |
|---|---|---|---|---|---|---|---|---|---|---|
| | | | | prior to | after | | | | | |
| | p/ | f/g/ | | stripping/ | stripping/ | H₂O(g)/ | AB/ | MHT/ | MCS/ | MEE/ |
| Example | bar | min | t/min | ppm | ppm | g | weight % | weight % | weight % | weight % |
| 2 | 4 | 4.1 | 100 | 5500 | <10 | 406 | 0.01 | 0.18 | 0.04 | 0.03 |
| 3 | 4 | 2.5 | 30 | 5500 | 66 | 75 | 0.02 | 0.2 | 0.03 | 0.02 |
| 4 | 4 | 0.8 | 360 | 5500 | <10 | 295 | 0.01 | 0 | 0.07 | 0.03 |
| 5 | 4 | 4.6 | 30 | 5500 | 50 | 138 | 0.02 | 0.12 | 0.03 | 0.03 |
| 6 | 4 | 4 | 360 | 5500 | <10 | 1442 | 0.06 | 0.12 | 0.07 | 0.06 |

### Example 7: Purification by Gas Stripping

After following a similar reaction procedure as described in Example 1,1254 g of the reaction product of reactor 1 was transferred to reactor 3 and azeotropically distilled. Afterwards, the remaining evanescent residuals were stripped with nitrogen gas stripping. Nitrogen gas was added with a pressure of 0.6 barg and a flow rate of 00.33 liter/m for a time period of 360 minutes. Afterwards, the sample was filtered, washed with ethanol and dried. Characterization by HPLC (AB, MHT, MCS, MEE) and GC (xylene) is provided in Table 3.

TABLE 3

| | Xylene | Xylene | | | | |
|---|---|---|---|---|---|---|
| | prior to | after | AB/ | MHT/ | MCS/ | MEE/ |
| Example | stripping/ppm | stripping/ppm | weight % | weight % | weight % | weight % |
| 7 | 5500 | <10 | 0.01 | 0.13 | 0.02 | 0.03 |

### Example 8: Purification by Steam Stripping

After following a similar reaction procedure as described in Example 1, 1254 g of the reaction product of reactor 1 was transferred to reactor 3 and azeotropically distilled. Afterwards, the remaining evanescent residuals were stripped with ethanol steam stripping. Ethanol steam was added with a pressure of 2.0 barg and a flow rate of 1.85 g/min for a time period of 300 minutes. Afterwards, the sample was filtered, washed with ethanol and dried. Characterization by HPLC (AB, MHT, MCS, MEE) and GC (xylene) is provided in Table 4.

TABLE 4

| | Xylene | Xylene | | | | |
|---|---|---|---|---|---|---|
| | prior to | after | AB/ | MHT/ | MCS/ | MEE/ |
| Example | stripping/ppm | stripping/ppm | weight % | weight % | weight % | weight % |
| 8 | 5500 | <10 | 0.01 | 0.16 | 0.03 | 0.20 |

A side effect of ethanol stripping is the higher amount of the mono ethylester of ethyl hexyl triazone (MEE) as side product, most likely resulting from transesterification.

### Example 9: Purification without any Stripping

The reaction product after azeotropic distillation was filtered, washed with ethanol and dried. Characterization by HPLC (AB, MHT, MCS, MEE) and GC (xylene) is provided in Table 5.

TABLE 5

| | Xylene | Xylene | | | | |
|---|---|---|---|---|---|---|
| | prior to | after washing and | AB/ | MHT/ | MCS/ | MEE/ |
| Example | stripping/ppm | drying/ppm | weight % | weight % | weight % | weight % |
| 9 | 5500 | 550 ppm | 0.24 | 0.19 | 0.17 | 0.06 |

As can be seen from comparing the results of Example 9 with the results of Examples 1 to 8, the xylene content in the product is significantly increased if no gas/steam stripping is performed. Surprisingly, also the amounts of impurities/side products (AB, MHT, MCS, MEE) present in the product are higher if no gas/steam stripping is performed.

1.-20. (canceled)

21. A process for preparing an s-triazine derivative of formula (I) 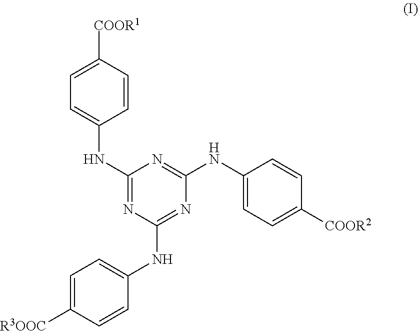
comprising reacting in a non-polar solvent selected from alkanes, aromatic solvents, and mixtures thereof
(i) a cyanuric halide; with
(ii) p-aminobenzoic acid esters 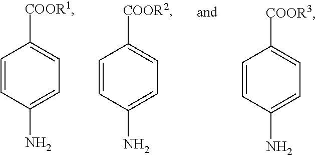
wherein
R¹, R², and R³ are each same or different and independently selected from the group consisting of hydrogen, alkali metal, ammonium, substituted ammonium, C₁-C₁₂-alkyl or polyoxyethylene; and
wherein
the reaction product is purified by gas/steam stripping.

22. The process according to claim 21, wherein
R²=R¹.

23. The process according to claim 21, wherein the s-triazine derivative is 2,4,6-trianilino-p-(carbo-2′-ethylhexyl-1′-oxy)-1,3,5-triazine having the following chemical formula (Ia):

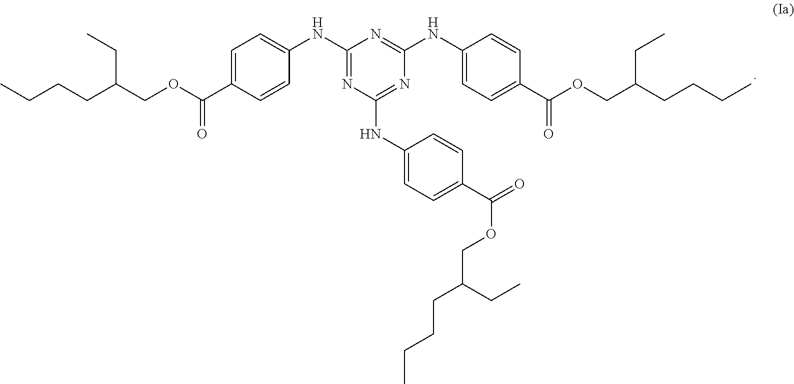 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

24. The process according to claim 21, wherein gas/steam stripping is performed by feeding the reaction product into a vessel or reactor and stripping off the non-polar solvent and optionally side products by adding water or a polar solvent or water steam or a steam of a polar solvent or a gas or a mixture thereof to the vessel or reactor.

25. The process according to claim 21, wherein gas/steam stripping is performed by adding water steam, ethanol steam, or nitrogen gas.

26. The process according to claim 21, wherein the temperature during steam/gas stripping is less than 200 °C or wherein the pressure of the steam/gas during steam/gas stripping is up to 100 bar.

27. The process according to claim 21, wherein, after stripping, the s-triazine derivative is dissolved in a polar solvent.

28. The process according to claim 21, wherein, after stripping, the s-triazine derivative is dissolved in a mixture of at least one C₁-C₄-alcohol with up to 10% by weight of water.

29. The process according to claim 21, wherein the reaction is performed in an aromatic solvent.

30. The process according to claim 21, wherein the reaction is performed at a temperature of from 75 °C to the boiling point of the used solvent for a time period of up to 48 h.

31. The process according to claim 21, wherein the cyanuric halide is selected from cyanuric chloride, cyanuric bromide and mixtures thereof.

32. The process according to claim 23, wherein the process further comprises preparing p-aminobenzoic acid-2-ethyl hexylester comprising reacting p-nitrobenzoic acid-2-ethyl hexylester with hydrogen in the presence of a catalyst.

33. The process according to claim 32, wherein the process further comprises preparing p-nitrobenzoic acid-2-ethyl hexylester comprising reacting p-nitrobenzoic acid with 2-ethylhexanol.

34. An s-triazine derivative of formula (I) having a purity of at least 98% by weight, wherein the derivative of formula (I) is tautomeric form I of 2,4,6-trianilino-p-(carbo-2′ethylhexyl-1′-oxy)-1,3,5-triazine and wherein the residual content of solvent is 100 ppm or lower, as determined by GC.

35. The s-triazine derivative according to claim 34, wherein the concentration of each one of p-aminobenzoic acid (AB), mono hydroxide of ethyl hexyl triazone (MHT), mono carboxylic acid of ethyl hexyl triazone (MCS), and mono ethylester of ethyl hexyl triazone (MEE), in the final product is below 0.2% by weight as measured by HPLC.

36. The s-triazine derivative according to claim 34, wherein the residual content of aromatic solvent is 100 ppm or lower, as determined by GC.

37. The s-triazine derivative according to claim 34, which is in the form of pourable or flowable particles.

38. An s-triazine derivative of formula (I) having a purity of at least 98% by weight, wherein the derivative of formula (I) is tautomeric form I of 2,4,6-trianilino-p-(carbo-2′ethylhexyl-1′-oxy)-1,3,5-triazine and wherein the residual content of solvent is 100 ppm or lower, as determined by GC, which is obtained by the process according to claim 21.

39. A light protecting agent comprising the s-triazine derivative of formula (I) according to claim 34.

40. A cosmetic preparation comprising the s-triazine derivative of formula (I) according to claim 34.
